(19) United States
(12) Patent Application Publication
(10) Pub. No.: US 2023/0366002 A1
(43) Pub. Date: Nov. 16, 2023

(54) RECOMBINANT YEAST FOR THE PRODUCTION OF OLIGOPEPTIDE

(71) Applicant: LESAFFRE ET COMPAGNIE, Paris (FR)

(72) Inventors: Simona Daly, Desio (MB) (IT); Stefano Galliani, Desio (MB) (IT); Immacolata Busiello, Sant'Antonino (CH); Matteo Grigis, Sant'Antonino (CH); Auro Roberto Tagliani, Desio (MB) (IT)

(73) Assignee: LESAFFRE ET COMPAGNIE, Paris (FR)

(21) Appl. No.: 18/246,607

(22) PCT Filed: Sep. 27, 2021
(86) PCT No.: PCT/EP2021/076468

§ 371 Date: Mar. 24, 2023

(30) Foreign Application Priority Data
Sep. 28, 2020 (IT) 102020000022846

(51) Int. Cl. C12P 21/02; C12N 1/18; C12N 15/90

(52) U.S. Cl. CPC C12P 21/02; C12N 1/185; C12N 15/905; C12R 2001/865

(57) ABSTRACT

The invention relates to a recombinant yeast wherein the PEP4 gene is inactivated. Said yeast is useful for the production of oligopeptides.

### FIELD OF INVENTION

The invention relates to genetically modified yeasts which are useful for the fermentative production of oligopeptides, in particular the production of γ-glutamyl-cysteinyl-glycine.

### BACKGROUND OF THE INVENTION

Glutathione (J. De Rey Pallade, Bull. Chem. Soc. France 31, 987-91, 1904) is a tripeptide (gamma-glutamyl-cysteinyl-glycine, often indicated as GSH) normally present in animal cells and involved as enzymatic substrate in numerous biochemical processes, mainly with the role of detoxifying agent (elimination of toxins in the form of a glutathionyl-derivative), metal chelating agent and reducing agent. In the latter role, it has considerable importance in reducing free radicals, and counteracting cell aging processes in general. Glutathione tends to oxidise, forming a dimer characterised by the presence of a disulphide bridge, and is often indicated as GSSG or “oxidised glutathione”. The two forms, oxidised and reduced, coexist in vivo. In view of its physiological importance, glutathione (in both reduced and oxidised form) is used as active ingredient in the formulation of pharmaceutical, nutraceutical and cosmetic products.

Glutathione can be prepared by chemical synthesis, but is usually produced by biotechnology, which is cheaper and gives rise to a product in optically pure form (Li et al., Appl. Microbiol. Biotechnol. 66, 233-42, 2004). The biomass is separated from the fermentation broths, and can then undergo lysis to release glutathione into the supernatant; the glutathione is then purified and isolated in solid form. The most common purification method involves a reaction with copper oxide or copper salts (U.S. Pat. No. 2,702,799) and a subsequent reaction with hydrosulphuric acid or salts thereof (CN106220708) or electrochemical reduction (EP2439312, EP2963156). Alternatively (EP1391517), glutathione can be purified solely by chromatography, thus avoiding the use of copper and H₂S on safety and environmental grounds.

Various examples in the literature describe the production of glutathione in wild-type or genetically modified yeasts of the genera Saccharomyces, Pichia and Candida (EP1391517, EP1512747, US2018/0135142) or in other microorganisms of bacterial origin, such as genetically modified Escherichia coli (EP2088153).

GSH can be accumulated in the biomass, or excreted into the supernatant (M. Rollini et al., Production of glutathione in extracellular form by Saccharomyces cerevisiae, Process Biochemistry 45, 441-445, 2010).

Biosynthesis of glutathione in S. cerevisiae involves 2 consecutive reactions. The first reaction, catalysed by the enzyme glutamate-cysteine ligase, gives rise to synthesis of γ-L-glutamyl-cysteine, starting with L-glutamate and cysteine. The second reaction is catalysed by the enzyme glutathione synthetase, which binds glycine to the dipeptide γ-L-glutamyl-cysteine, thus forming glutathione, or the tripeptide γ-L-glutamyl-L-cysteinylglycine (γ-Glu-Cys-Gly). The biosynthesis can be increased with molecular biology techniques in recombinant strains.

In competition with biosynthesis methods, there are also biodegradation methods, which prevent the accumulation of glutathione in the biomass; glutathione is metabolised and reconverted to the three constituent amino acids, by reactions catalysed by the respective enzymes. In the main known degradation pathway, the first enzyme, γ-glutamyl transpeptidase (encoded by gene ECM38), hydrolyses γ-L-glutamyl, leading to formation of the dipeptide cysteinyl-glycine, and releasing glutamic acid; the second enzyme, cysteinylglycine peptidase, hydrolyses the dipeptide, releasing cysteine and glycine. Glutathione degradation therefore gives rise to formation of the dipeptide cysteinyl-glycine (Cys-Gly).

A second GSH degradation pathway has been postulated in recombinant Saccharomyces cerevisiae strains wherein the main pathway had been deleted (Kumar et al., FEMS Microbiology Lett 219, 187-94, 2003). Said second pathway was subsequently identified, and proved to be catalysed by the “dug complex”, comprising three enzymes, encoded by genes DUG1, DUG2 and DUG3. In particular, it involves the combined action of a peptidase (DUG2) and glutamine amidotransferase (DUG3), together with a protease (DUG1, also called dipeptidase) (Bachhawat et al., Genetics 175, 1137-51, 2007).

For industrial production it is important to limit glutathione degradation at the end of fermentation, when the concentration in the biomass has reached the maximum level; the treatment of industrial amounts of biomass necessarily requires several hours' processing time, during which degradation of the product involves a reduction in yield and complicates purification. It would therefore be reasonable to consider preventing degradation by inactivating both the gamma-GT pathway, encoded by gene ECM38, and the DUG pathway, encoded by genes DUG1, DUG2 and DUG3, as described in Kumar et al., J Biol Chem 287, 4552-61, 2012.

However, double deletion of said two glutathione degradation pathways is insufficient, as described below.

### DESCRIPTION OF THE INVENTION

It has now been discovered that a third glutathione degradation pathway exists. In fact, recombinant yeasts carrying the double deletion of the two above-mentioned degradation pathways are still able to degrade glutathione; the GSH content of the biomass at the end of fermentation tends to fall rapidly, much faster than is attributable to chemical (spontaneous) degradation. Said degradation is therefore enzymatic, and obviously has an adverse effect on the yield obtainable from industrial production.

It has now surprisingly been found that an enzyme already known and normally present in yeasts, namely a protease known as aspartyl protease or proteinase A and encoded by the PEP4 gene (Ammerer et al., Mol Cell Biology, 6, 2490-2499, 1986), degrades glutathione by hydrolysing it into glycine and γ-L-glutamyl-cysteine (γ-Glu-Cys).

Proteinase A is a proteolytic enzyme present in the vacuoles of S. cerevisiae, which has long been known and classified as pepsin-like aspartyl proteinase; aspartyl proteinases are widely distributed in vertebrates, fungi, plants and retroviruses, with different functions and different ranges of optimum pH. The difference in functions is reflected in the low homology between the genome sequences encoding the enzymes belonging to the family (Parr et al., Yeast, 2007).

In beer manufacture, S. cerevisiae proteinase A is involved in the degradation of the proteins that contribute to froth formation; deletion of the PEP4 gene gives rise to better quality and greater stability of the froth on the beer (Wang et al., Int J of Food Microbiol, 2007; CN1948462).

The inactivation of proteinase A is also described in a recombinant strain of Pichia pastoris, used for the production of human parathyroid hormone, to prevent proteolytic degradation of said parathyroid hormone (Wu et al., J Ind Microbiol Biotechnol, 2013).

However, the action that proteinase A can perform on glutathione, causing its degradation with formation of γ-Glu-Cys dipeptide, has never been described. It would therefore not have been expected that inactivating the PEP4 gene would improve the stability of glutathione in the biomass.

In fact, it has been demonstrated that the presence of proteinase A in active form exerts an adverse effect on the accumulation of glutathione, which is partly hydrolysed, in the biomass. The product of hydrolysis is not cysteinyl-glycine (Cys-Gly) dipeptide, as expected according to the glutathione degradation pathway, but γ-L-glutamyl-cysteine dipeptide (γ-Glu-Cys). The presence of proteinase A in active form therefore has an adverse effect on the stability of the glutathione produced. This is observed in particular in the period between the end of the fermentation process and the subsequent stages of glutathione lysis, extraction and purification. Under said process conditions, a reduction in the glutathione content of the biomass and a simultaneous increase in the γ-L-glutamyl-cysteine dipeptide (γ-Glu-Cys) content is observed.

γ-L-glutamyl-cysteine dipeptide (γ-Glu-Cys) is also an impurity difficult to separate from glutathione, as it has chemical characteristics (the presence of a free thiol, with reducing and metal-complexing capacity) and biochemical characteristics (molecular weight, isoelectric point) very similar to those of glutathione. The presence of high concentrations of γ-Glu-Cys dipeptide can therefore interfere with the glutathione purification process. A further advantage of deletion of the PEP4 gene is therefore a more efficient purification process of the glutathione obtained from a strain of yeast.

The present invention consists of a strain of yeast wherein the PEP4 gene functionality has been reduced, e.g. by altering the gene structure or expression, or it has been suppressed by partial or complete gene deletion; the proteinase A enzyme is therefore not produced, or is produced in form that is not catalytically active. This increases the stability over time of the glutathione produced by the cells and contained in the biomass, and maintains a low concentration of γ-Glu-Cys dipeptide, benefiting the quality of the product and the process yield.

A further way of inactivating the enzyme is to add protease inhibitors, more specifically aspartyl protease inhibitors. Said substances inhibit the activity of the enzyme, which in turn can no longer exert its glutathione degradation activity.

The end result of said actions is therefore to increase the efficiency of the glutathione manufacturing process.

### DETAILED DESCRIPTION OF THE INVENTION

The subject of the present invention is a recombinant microorganism able to produce glutathione, characterised in that the PEP4 gene, encoding proteinase A, has been inactivated in said microorganism.

One aspect of the invention requires said microorganism to be a yeast, such as a haploid or diploid yeast. In a particular embodiment of the invention, said microorganism is a diploid yeast wherein both copies of the PEP4 gene have been inactivated.

According to the invention, the PEP4 gene can be inactivated by total or partial deletion thereof, or by mutagenesis or insertion of exogenous DNA, such as a selection marker using a homologous recombination process. In any event, inactivation of the gene abolishes or reduces the expression of proteinase A or gives rise to expression of a non-functional proteinase A.

The invention demonstrates that:
inactivation of the PEP4 gene improves the stability of the glutathione produced, while its titer value remains stable at room temperature
inactivation of the PEP4 gene reduces the presence of γ-glutamyl-cysteine dipeptide (γ-Glu-Cys) under fermentation conditions
degradation of glutathione to γ-L-glutamyl-cysteine (γ-Glu-Cys) is also reduced under non-fermentative conditions, during purification of the product (downstream).

In a preferred embodiment, the invention provides a genetically modified yeast by inactivation of the PEP4 gene and of at least one gene involved in glutathione degradation via the gamma-GT or DUG pathway. Said gene involved in glutathione degradation via the gamma-GT or DUG pathway is preferably selected from ECM38, DUG1, DUG2 and DUG3.

In yeast, a specific target gene can be inactivated by a recombination mechanism that replaces a given gene with another (marker) gene, such as genes that confer resistance to an antibiotic or another toxic substance, auxotrophic markers or other genes.

To facilitate the subsequent steps, the marker genes are constructed so that they are flanked by short repeated sequences recognised by specific recombinases that catalyse the removal of the DNA fragment, and then eliminate the marker gene. For example, the sequences LoxP or LoxR, recognised by recombinases called “Cre” or “R”, can be used in this way, and there are numerous alternative methods which are substantially equivalent.

According to the invention, other genetic modifications of the microorganism, designed to increase the biosynthesis capacity of glutathione, can also be effected, for example by inserting one or more copies of the GSH1 and GSH2 genes, as described below.

In a particular embodiment of the invention, the recombinant microorganism obtained by inactivation of the PEP4 gene is a microorganism belonging to the species Saccharomyces cerevisiae. The PEP4 gene of S. cerevisiae, which consists of 1218 nucleotides (NCBI Reference Sequence: NM 001183968.1), is located in the genome of S. cerevisiae in chromosome XVI, 2 copies of which are present in the diploid cell.

Although according to a representative embodiment of the invention the recombinant microorganism able to produce glutathione derives from S. cerevisiae, any microorganism belonging to the yeast group can be used. Examples of said microorganisms include yeasts belonging to the genera Candida, such as C. utilis, Pichia, such as P. pastoris, Kluyveromyces, such as K. lactis, and Schizosaccharomyces, such as S. pombe.

The yeast from which the recombinant microorganism according to the invention derives is preferably S. cerevisiae, diploid strain GN2361 or GN2362 or GN2373, which naturally contains the PEP4 gene, encoding a protein with protease activity. S. cerevisiae strains GN2361, GN2362 and GN2373 originate in turn from S. cerevisiae strain BY4742, held in the American Type Culture Collection (ATCC), assigned code ATCC 201389. Starting from strain BY4742, with engineering activities conducted according to the known art, all the previously known glutathione degradation pathways, encoded by the ECM38, DUG2 and GCG1 genes, were inactivated (Ganguli et al. 2007, Genetics, and Baudouin-Cornu et al. 2012, J. Biol. Chem.).

However, despite the deactivation of the metabolic pathways, the biomasses obtained from said strains gave rise to glutathione degradation in the downstream stages; during purification of the product, a reduction in glutathione content was observed, together with an increase in an impurity, later identified as γ-glutamyl-cysteine (γ-Glu-Cys).

Another gene, not connected with the biosynthesis pathway or the known metabolic pathways of glutathione, namely the PEP4 gene, was then inactivated, obtaining biomasses wherein the biochemical degradation of glutathione to γ-Glu-Cys is eliminated; said biomasses with improved stability are compatible with industrial processing times, and therefore offer the advantage of better product purification.

Surprisingly, said biomasses also exhibit lower presence of γ-Glu-Cys in the broths at the end of fermentation. By fermenting the original strains containing the PEP4 gene, and the corresponding derivative strains devoid of proteinase A, under the same conditions, a better ratio between the desired product (glutathione) and the undesirable product (γ-Glu-Cys) is obtained in the latter strains.

The present invention demonstrates that inactivation of the PEP4 gene gives rise to biomasses of better quality in the fermentative production of glutathione, and simultaneously promotes the industrial processability of the biomasses.

Another yeast from which the recombinant microorganism according to the invention can derive is S. cerevisiae, haploid strain GN2357, wherein the PEP4 gene is located in the genome, again in chromosome XVI; however, only one copy thereof is present in the haploid cell.

Inactivation of the PEP4 gene in the recombinant diploid or haploid microorganism can be achieved by replacing the nucleotide sequence of the gene with the sequence of an exogenous gene that confers resistance to G418, an aminoglycoside antibiotic with a structure similar to gentamicin. The inserted exogenous gene is subsequently removed by means of a recombination process in the yeast cells. The result is deletion of the PEP4 gene and loss of its function.

The method used to obtain a recombinant strain of S. cerevisiae able to accumulate glutathione with greater stability due to inactivation of the PEP4 gene can generally be applied to other yeasts whose glutathione stability is to be improved.

In an embodiment of the present invention, glutathione degradation and γ-Glu-Cys production are reduced in Pichia pastoris strains; in particular, under the same experimental conditions, the strain devoid of the PEP4 gene exhibits lower production of γ-Glu-Cys, even over long periods.

The following examples illustrate the invention in greater detail.

### BRIEF DESCRIPTION OF FIGURES

FIG. 1: deletion of the DUG2 gene by substitution with the URA3 gene of K. lactis flanked by 2 repeated loxP sequences.

FIG. 2: deletion of the PEP4 gene by substitution with the KanMX4 gene flanked by two FRT sequences and two regions homologous with the PEP4 gene.

FIG. 3: stability of GSH—the graph shows the level of γ-Glu-Cys dipeptide present in the biomasses of S. cerevisiae at different times

FIG. 4: stability of GSH—the graph shows the level of γ-Glu-Cys dipeptide present in the biomasses of P. pastoris at different times.

### EXAMPLE 1 (COMPARATIVE)

The yeast Saccharomyces cerevisiae strain NCYC2958 is cultured as described in EP1391517, Example 3; at the end of fermentation the yeast is centrifuged, and then washed in the centrifuge with demineralised water. The resulting biomass is dispersed in 10 volumes of an aqueous solution containing glucose and the other nutrients described, to increase the reduced glutathione content of the biomass; at the end of said procedure the whole broth is centrifuged and the biomass is washed with demineralised water to eliminate the supernatant.

The GSH-enriched yeast biomass then undergoes thermoacid lysis followed by microfiltration through ceramic membranes with a porosity of 0.2 microns, as described in Example 1 of EP1391517. The resulting almost clear solution is applied on a column of ion-exchange resin, then on adsorbent resin, and finally concentrated by nanofiltration, as described in paragraphs [0060] and [0061] of said patent.

Reduced glutathione in powder form is obtained from the purified aqueous solution by spray-drying; the resulting product complies with the purity specifications laid down in the European Pharmacopoeia.

### EXAMPLE 2 CONSTRUCTION OF A RECOMBINANT STRAIN OF S. CEREVISIAE WITH DELETION OF ECM38 AND DUG2

Starting with strain BY4742, the previously known glutathione degradation pathways, encoded by the ECM38 and DUG2 genes, were inactivated by engineering activities conducted as described in the prior art (Ganguli et al. 2007, Genetics, Baudouin-Cornu et al., 2012, J Biol Chem).

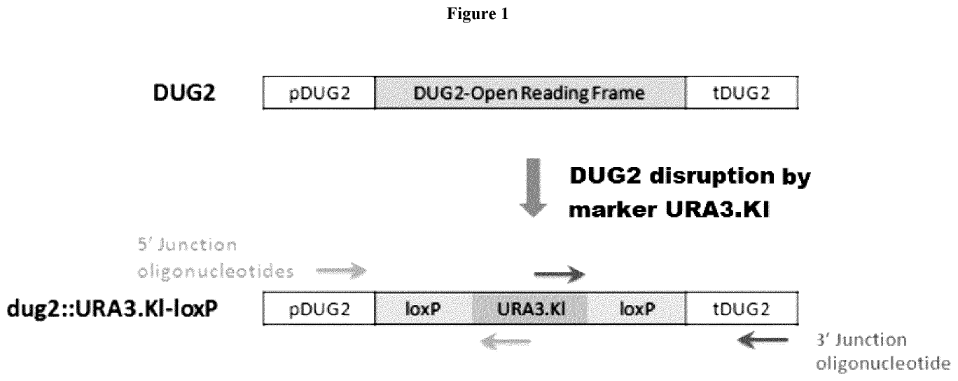

The DUG2 gene was eliminated in strain BY4742 by substitution with the URA3 gene of Kluyveromyces lactis (homologue of the URA3 gene of Saccharomyces cerevisiae), flanked by 2 repeated loxP sequences (FIG. 1).

A DNA fragment comprising the LoxP-URA3-LoxP cassette and flanked by regions 5′ and 3′ of the DUG2 gene was used to transform strain BY4742; the transformants, selected for their ability to grow on uracil-free synthetic medium, were purified and analysed to confirm the substitution of the DUG2 gene with the URA3 marker. An expression cassette containing the GSH1 and GSH2 genes, which catalyse the 2 enzymes required for glutathione biosynthesis, was then inserted in the locus that initially contained the DUG2 gene.

The ECM38 gene was eliminated (in the strain already deleted for DUG2), by substitution with the LEU2 gene marker of Kluyveromyces lactis (homologue of the LEU2 gene of Saccharomyces cerevisiae), following the same steps as described for DUG2. Finally, subsequent recombinase induction eliminated the 2 URA3 and LEU2 marker genes.

A strain was thus obtained which, as well as having the DUG2 and ECM38 genes (responsible for glutathione degradation) deleted, also contains additional copies of the genes GSH1 and GSH2 that increase glutathione biosynthesis and production.

### EXAMPLE 3 CONSTRUCTION OF A RECOMBINANT PEP4-DELETED STRAIN OF S. CEREVISIAE

The microorganism of the previous example is transformed with a DNA fragment containing a sequence (KanMX4) that confers resistance to compound G418. As a result of the transformation, said sequence is inserted in the place of the endogenous PEP4 gene, thereby inducing its knockout. For haploid strains, the result is the knockout of the only copy of the PEP4 gene existing in the genome of the microorganism; for diploid yeasts, the process is repeated to eliminate the second copy of the PEP4 gene too.

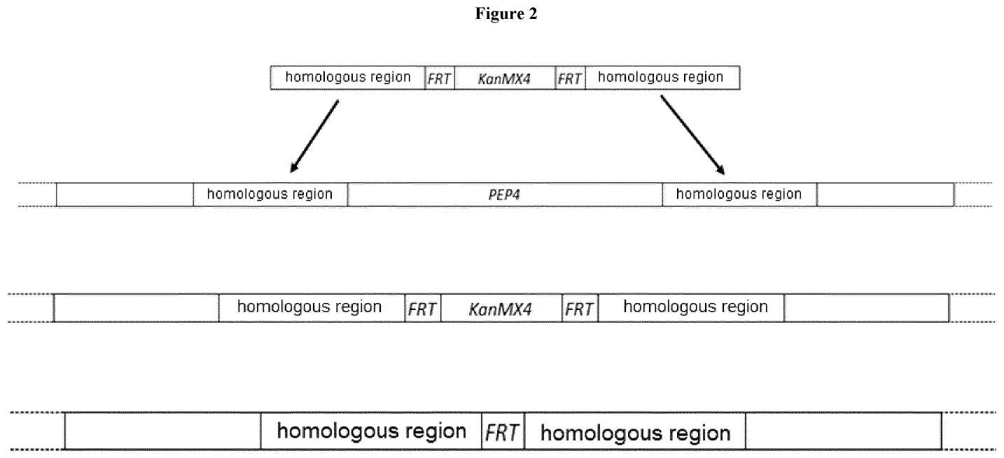

The DNA fragment used for the transformation contains the sequence of the KanMX4 gene (810 bp), flanked by two FRT (Flippase Recognition Target) recombination sequences and two regions homologous with the PEP4 gene (first part of FIG. 2), which serve to allow site-specific recombination of the fragment in the PEP4 locus.

The KanMX4 gene is obtained by amplification from plasmid pWKW (Storici et al. 1999, Yeast 15:271-283), using the binding sites of primers P1 and P2.

Two different DNA fragments, each of which is obtained via a specific pair of oligonucleotides, are used to knock out each of the two copies of the PEP4 gene present in the genome of the microorganism.

The following oligonucleotides are used for amplification of the first fragment and knockout of the first copy of the PEP4 gene:

FOR1
(SEQ ID NO: 1)
TTGTTATCTACTTATAAAAGCTCTCTAGATGGCAGAAAAGGATAGGGCG
GAGAAGTAAGAAAAGTTTAGCAAAAATAGGCGTATCACGAG
REV1
(SEQ ID NO: 2)
AAAGAAAAAAAAAAAGCCTAGTGACCTAGTATTTAATCCAAATAAAATT
CAAACAAAAACCAAAACTAACTCGATGATAAGCTGTCAAAC

The following oligonucleotides are used for amplification of the second fragment and knockout of the second copy of the PEP4 gene:

FOR2
(SEQ ID NO: 3)
TCAAATTGCTTTGGCCAAACCAACCGCATTGTTGCCCAAATCGTAAATA
GAATAGTATTTACGCAAGAAGAAAAATAGGCGTATCACGAG
REV2
(SEQ ID NO: 4)
ATGTTCAGCTTGAAAGCATTATTGCCATTGGCCTTGTTGTTGGTCAGCG
CCAACCAAGTTGCTGCAAAAGTCGATGATAAGCTGTCAAAC

Fragments 1 and 2 thus obtained are purified and used for transformation of the microorganism by the lithium acetate method (Kawai et al. 2010 Bioeng bugs 1(6) 395-403).

The yeast is transformed with fragment 1 and plated on YPD medium containing selection agent G418; 3 G418-resistant colonies are obtained and isolated. To verify the transformation and recombination of fragment 1 in the PEP4 locus, the 3 colonies are analysed by PCR amplification using the following primers and conditions:

| (SEQ ID NO: 5) | |
|---|---|
| F1 | TGATTTCAAATGTTTCTAGAGCGCA |
| (SEQ ID NO: 6) | |
| R1 | AATGCTGAAATTGGGGCCAA |
| (SEQ ID NO: 7) | |
| F2 | GCGTTCAAGTAATTTGTCAATGGAA |
| (SEQ ID NO: 8) | |
| R2 | TTTGAGAAGCCTACCACGTAAGG |
| (SEQ ID NO: 9) | |
| K1 R1 | TACAATCGATAGATTGTCGCAC |
| | works with F1 and R1 |
| (SEQ ID NO: 10) | |
| K2 F2 | AGTCGTCACTCATGGTGATT |
| | works with F2 and R2 |

The PCR products are analysed by 0.8% gel electrophoresis which identifies a 953 bp fragment and a 720 bp fragment, as expected.

The 3 transformants are inoculated into liquid YPD medium and left to grow under stirring at 200 rpm, 30° C., for 20 hours. During cell incubation, the endogenous recombination system of S. cerevisiae Flp/FRT is activated, leading to excision of the heterologous KanMX4 gene (Park Y N et al. Yeast 28(9) 673-681, 2011). Each of the 3 cultures, suitably diluted, is plated on YPD medium (in the absence of selective agent G418). The colonies grown on the plates are then transferred by replica-plating to plates of YPD+G418 medium. The colonies that fail to grow even on said plates are those which, due to the Flp/FRT recombination, have lost the heterologous KanMX4 gene. Said colonies are isolated from the original YPD plates and analysed by PCR using the following primers and conditions:

| (SEQ ID NO: 11) | |
|---|---|
| F1 | TGATTTCAAATGTTTCTAGAGCGCA |
| (SEQ ID NO: 12) | |
| R2 | TTTGAGAAGCCTACCACGTAAGG |

The PCR products are analysed by 0.8% gel electrophoresis which identifies a 600 bp fragment, as expected, confirming the knockout of the first copy of the PEP4 gene.

### EXAMPLE 4 CONSTRUCTION OF A RECOMBINANT DIPLOID STRAIN OF S. CEREVISIAE

Construction of strains GN2363 (from GN2361), GN2364 (from GN2362) and GN2376 (from GN2373). The procedure is conducted on the original strains GN2361, GN2362 and GN2373 as described in experiment 2, obtaining the corresponding PEP4-deleted strains: GN2363, GN2364 and GN2376.

The procedure proved replicable and applicable to various strains of yeast.

Yeast GN2363 is deposited and registered at the Collection Nationale de Cultures de Microorganismes—Institut Pasteur (Paris, International Depositary Authority under the Budapest Treaty), under registration number CNCM 1-5574.

Yeast GN2364 is deposited and registered at the Collection Nationale de Cultures de Microorganismes—Institut Pasteur (Paris, International Depositary Authority under the Budapest Treaty), under registration number CNCM 1-5575.

### EXAMPLE 5 CULTIVATION OF YEAST ON A LABORATORY SCALE AND GSH STABILITY TEST

Strains GN2361 and GN2363 (original and recombinant) are cultured under the same conditions using a growth process in liquid culture, in an Erlenmeyer flask, comprising a vegetative stage followed by a productive stage.

The vegetative stage is obtained by inoculating 0.5 ml of a stock of cells (frozen and stored at −80° C.) into 20 ml of vegetative medium (1% yeast extract, 2% peptone, 2% glucose). The cultures are left to grow at 28° C. for 16 hours under stirring at 200 rpm. At the end of the incubation period, 10 ml of the vegetative culture is inoculated into 90 ml of productive medium (2% yeast extract, 8% glucose, 0.2% cysteine, 0.2% glycine, 0.2% L-glutamate). The cultures are left to grow at 28° C. for 48 hours under stirring at 250 rpm.

At the end of the incubation period the culture is divided into 2 equal aliquots to obtain 2 equal samples for use in the stability tests.

For each culture, one of the aliquots is immediately subjected to heat lysis, and its glutathione and γ-Glu-Cys dipeptide content analysed by the HPLC method. The second aliquot is incubated at 25° C. for 24 hours. After the incubation period the sample is subjected to heat lysis, and its glutathione and γ-Glu-Cys dipeptide content analysed.

The results are set out in Table 1, which shows the mean value obtained from 4 independent samples.

TABLE 1

| | Time | GSH | % GSH | γ-GC | % γ-GC |
|---|---|---|---|---|---|
| strain | (hours) | mg/L | residue | mg/L | increase |
| GN2361 | 0 | 1083 | 100 | 42.7 | 0 |
| | 24 | 1025 | 95 | 90.7 | 112 |
| GN2363 | 0 | 949 | 100 | 12.0 | 0 |
| | 24 | 927 | 98 | 14.3 | 19 |

The results demonstrate that PEP4-deleted strain GN2363 produces a smaller amount of γ-Glu-Cys dipeptide, and this remains constant even after 24 hours' incubation at 25° C. Original strain GN2361 (which contains the PEP4 gene) presents a 112% increase in the amounts of γ-Glu-Cys dipeptide, as well as exhibiting greater GSH degradation (95% GSH residue vs 98%).

### EXAMPLE 6 CULTIVATION OF YEAST ON A LABORATORY SCALE, AND TEST OF GLUTATHIONE STABILITY IN THE BIOMASS

Strains GN2362 and GN2364 (original and recombinant) are cultured, and the stability test on the GSH and γ-Glu-Cys dipeptide content conducted, on a laboratory scale, using the same procedures as described in Example 4.

The results are set out in Table 2, which indicates the mean value obtained from 4 independent samples.

TABLE 2

| | Time | GSH | % GSH | γ-GC | % γ-GC |
|---|---|---|---|---|---|
| Strain | (hours) | mg/L | residue | mg/L | increase |
| GN2362 | 0 | 1147 | 100 | 42.1 | 0 |
| | 24 | 1095 | 95 | 79.3 | 88 |
| GN2364 | 0 | 924 | 100 | 13.6 | 0 |
| | 24 | 890 | 96 | 15.5 | 14 |

The results demonstrate that strain GN2364 (Apep4 corresponding to GN2362) produces a smaller amount of γ-Glu-Cys dipeptide than the parent strain. The increase in γ-Glu-Cys is considerably lower in strain GN2364 than parent strain GN2362 (14% vs 88% after 24 hours' incubation).

### EXAMPLE 7 CULTIVATION OF YEAST ON A LABORATORY SCALE AND GSH STABILITY TEST

Strains GN2373 and GN2376 (original and recombinant) are cultured, and the stability test on the GSH and γ-Glu-Cys dipeptide content conducted, on a laboratory scale, using the same procedures as described in Example 4.

The results are set out in Table 3, which indicates the mean value obtained from 4 independent samples.

TABLE 3

| | Time | GSH | % GSH | γ-GC | % γ-GC |
|---|---|---|---|---|---|
| Strain | (hours) | mg/L | residue | mg/L | increase |
| GN2373 | 0 | 1056 | 100 | 41.5 | 0 |
| | 24 | 1054 | 100 | 71.0 | 71 |
| GN2376 | 0 | 926 | 100 | 20.9 | 0 |
| | 24 | 918 | 99 | 20.1 | −3 |

The results demonstrate that recombinant strain GN2376 produces a smaller amount of γ-Glu-Cys dipeptide, which remains constant after 24 hours' incubation at 25° C. Original strain GN2373 (which still contains the PEP4 gene) presents a 71% increase in the amount of γ-Glu-Cys dipeptide.

### EXAMPLE 8 CULTIVATION OF HAPLOID S. CEREVISIAE ON A LABORATORY SCALE AND GSH STABILITY TEST

Strains GN2357 and GN2357-Apep4 are cultured, and the stability test on the GSH and γ-Glu-Cys dipeptide content conducted, on a laboratory scale, using the same procedures as described in Example 4.

The results are set out in Table 4, which shows the mean value obtained from 4 independent samples.

TABLE 4

| | Time | GSH | % GSH | γ-GC | % γ-GC |
|---|---|---|---|---|---|
| Strain | (hours) | mg/L | residue | mg/L | increase |
| GN2357 | 0 | 594.1 | 100 | 77.0 | 0 |
| | 24 | 565.7 | 95 | 112.5 | 46 |
| GN2357-Δpep4 | 0 | 683.1 | 100 | 20.8 | 0 |
| | 24 | 661.4 | 97 | 21.3 | 2 |

The results demonstrate that strain GN2357-Apep4 produces a smaller amount of γ-Glu-Cys dipeptide, which remains constant even after 24 hours' incubation at 25° C. Instead, the strain which still contains the PEP4 gene presents a 46% increase in the amount of γ-Glu-Cys dipeptide.

### EXAMPLE 9 CULTIVATION OF YEAST ON A PILOT SCALE AND GSH STABILITY TEST

Strains GN2361 and the corresponding GN2363 (recombinant Apep4) are cultured by a growth process in liquid medium, comprising a pre-vegetative stage and a vegetative stage in an Erlenmeyer flask, and a fermentative stage and productive stage in a bioreactor.

The pre-vegetative stage is conducted as described in Example 4.

The vegetative stage is conducted by transferring 0.1 ml of pre-vegetative culture into 400 ml of vegetative medium (1% yeast extract, 2% peptone, 2% glucose) in an Erlenmeyer flask. The culture is incubated at 28° C. for 24 hours under stirring at 240 rpm.

The fermentative stage is conducted by transfer into a 7 L bioreactor containing productive medium (yeast extract, glucose, ammonium, phosphate, sulphate and vitamin and mineral supplements) at 28° C., gassed (1-2 VVM air) and stirred (600-1200 rpm).

The biomass of the fermentative culture is harvested, concentrated to half its volume by centrifugation, and reintroduced into a 7 L bioreactor containing productive medium (glucose, ammonium, phosphate, sulphate, cysteine, glycine and glutamic acid) at 28° C., gassed (1 VVM air) and stirred (600 rpm).

At the end of the incubation period the culture is divided into 4 equal aliquots to obtain 4 equal samples for use in the stability tests.

For each culture, one aliquot is immediately subjected to heat lysis, and its glutathione and γ-Glu-Cys dipeptide content is analysed by the HPLC method. The remaining 3 aliquots are incubated at 25° C. for 24, 48 and 72 hours respectively. After each incubation period the sample is subjected to heat lysis, and its glutathione and γ-Glu-Cys dipeptide content is analysed.

The results are set out in Table 1, which shows the data obtained with the original strain GN2361 and the data from two independent tests with the corresponding genetically modified yeast GN2363.

TABLE 5

| Strain | Time (h) | GSH % | γ-GC (mg/l) | γ-GC % |
|---|---|---|---|---|
| GN2361 | 0 | 100 | 1326 | 100 |
| | 24 | 68 | 2983 | 225 |
| | 48 | 44 | 2932 | 221 |
| GN2363 trial 1 | 0 | 100 | 100 | 100 |
| | 24 | 92 | 130 | 130 |
| | 48 | 71 | 135 | 135 |
| GN2363 trial 2 | 0 | 100 | 180 | 100 |
| | 24 | 93 | 278 | 154 |
| | 48 | 84 | 270 | 150 |

GSH and γ-GC: HPLC titer of glutathione and γ-glutamyl-cysteine

The data demonstrate increased stability of glutathione in the genetically modified biomasses, with less overall degradation (% titer reduction) and enzymatic degradation almost eliminated (limited increase of γ-GC).

### EXAMPLE 10 CULTIVATION OF YEAST ON A PILOT SCALE AND GSH STABILITY TEST

Strain GN2362 and its corresponding strain GN2364 (modified Apep4) are cultured as described in Example 9.

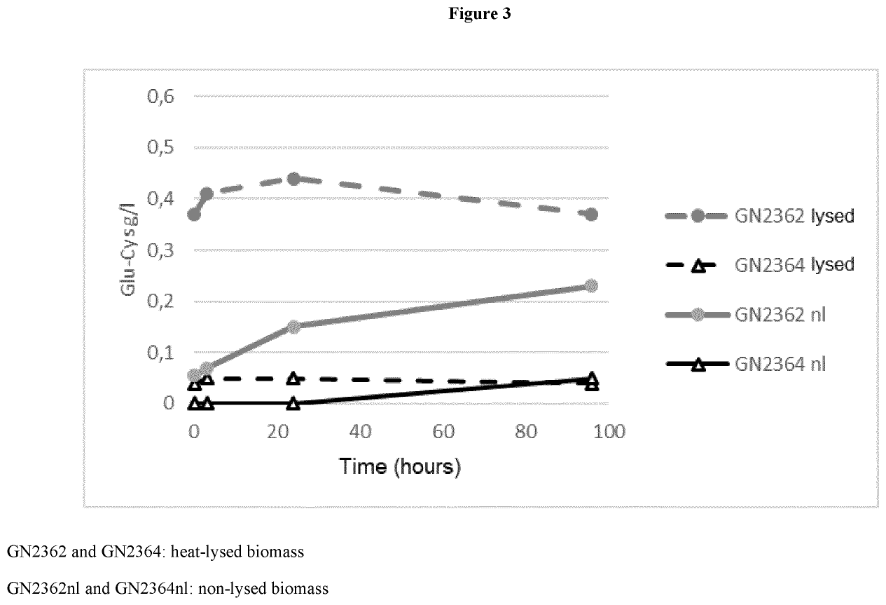

The results are set out in the table below and in FIG. 3.

TABLE 6

| Strain | Time (h) | GSH % | γ-GC (mg/l) | γ-GC % |
|---|---|---|---|---|
| GN2362 | 0 | 100 | 1503 | 100 |
| | 24 | 70 | 4170 | 277 |
| | 48 | 56 | 4451 | 296 |
| GN2364 | 0 | 100 | 548 | 100 |
| | 24 | 99 | 428 | 78 |
| | 48 | 90 | 300 | 55 |

GSH and γ-GC: HPLC titer of glutathione and γ-glutamyl-cysteine

The data demonstrate the greater stability of glutathione in the genetically modified biomasses, with less overall degradation (% titer reduction) and enzymatic degradation almost eliminated (limited increase of γ-GC). γ-GC degrades slowly by a chemical process.

In GN2362, cell lysis rapidly releases proteinase A, while the growth of γ-Glu-Cys is faster, then slowly declines due to spontaneous degradation.

In GN2364 the growth of γ-Glu-Cys is slower with both whole and lysed cells.

### EXAMPLE 11 FERMENTATION OF PICHIA PASTORIS AND GLUTATHIONE STABILITY TEST

The strains Pichia pastoris X-33 (which contains PEP4), SMD1168H (which does not contain PEP4) and GN2364 (recombinant S. cerevisiae, described above) are cultured in a suitable medium for 48 h, at 28° C. and 250 rpm. At the end of fermentation the cell biomass is harvested by centrifugation and resuspended in dH₂O, obtaining one suspension for each strain.

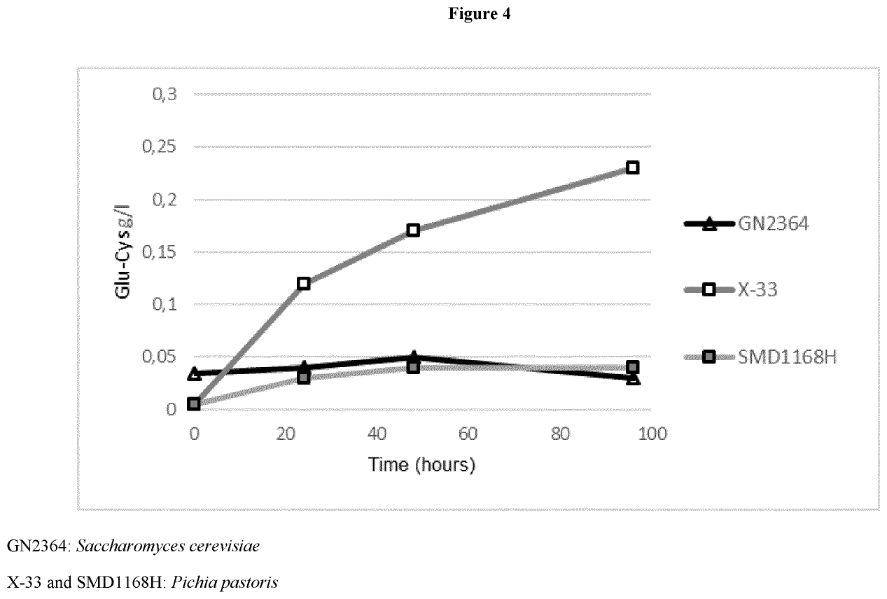

A stock solution of glutathione in dH₂O is prepared at the concentration of 150 g/l. One aliquot of the stock solution is added to the cell biomass suspension, obtaining a final GSH concentration of 10 g/l. The cell biomass with added GSH is divided into 1.5 ml aliquots, which are incubated at a controlled temperature of 25° C., with stirring at 900 rpm. The formation of γ-Glu-Cys is monitored for up to 96 hours, analysing samples incubated for different times by HPLC analysis. The resulting data are set out in FIG. 4.

The data demonstrate the degradation of glutathione to give γ-Glu-Cys by the Pichia X-33 strain, whereas the two yeasts devoid of the PEP4 gene, Pichia SMD1168H and Saccharomyces GN2364, exhibit the same behaviour and do not increase the production of γ-Glu-Cys.

1. Yeast genetically modified by inactivation of the PEP4 gene and of at least one gene involved in glutathione degradation through the γ-GT or DUG pathway.

2. Yeast according to claim 1, wherein said gene involved in glutathione degradation through the γ-GT or DUG pathway is selected from ECM38, DUG1, DUG2 and DUGS.

3. Yeast according to claim 1, wherein said inactivation of the PEP4 gene is obtained by total or partial gene deletion, or by mutagenesis or by insertion of exogenous DNA.

4. Yeast according to claim 1, which is haploid or diploid and wherein one or both the PEP4 gene copies are inactivated.

5. Yeast according to claim 1, which belongs to a genus selected from Saccharomyces and Pichia.

6. Yeast according to claim 5, which is selected from S. cerevisiae and P. pastoris.

7. Yeast according to claim 1, which is further genetically modified by introduction of one or more additional copies of the GSH1 or GSH2 gene.

8. Yeast according to claim 1, which belongs to the species S. cerevisiae, a strain whereof is deposited at CNCM—Institute Pasteur with registration number CNCM 1-5574 or CNCM 1-5575.

9. Fermentative process for the production of glutathione, comprising the following steps:
(i) culturing a yeast as defined in claim 1, thereby forming a biomass; and
(ii) separating and purifying glutathione from the biomass.

10. Biomass for glutathione production, which is obtainable by the process according to claim 9, step (i).

11. (canceled)

12. Yeast according to claim 3, wherein said inactivation of the PEP4 gene is obtained by homologous recombination with exogenous DNA.


SEQUENCE LISTING (12 sequences)

SEQ ID NO: 1 - DNA, 90 bp, Unknown
  artificial oligonucleotide
ttgttatctacttataaaagctctctagatggcagaaaaggatagggcggagaagtaagaaaagtttagcaaaaataggcgtatcacgag

SEQ ID NO: 2 - DNA, 90 bp, Unknown
  artificial oligonucleotide
aaagaaaaaaaaaaagcctagtgacctagtatttaatccaaataaaattcaaacaaaaaccaaaactaactcgatgataagctgtcaaac

SEQ ID NO: 3 - DNA, 90 bp, Unknown
  artificial oligonucleotide
tcaaattgctttggccaaaccaaccgcattgttgcccaaatcgtaaatagaatagtatttacgcaagaagaaaaataggcgtatcacgag

SEQ ID NO: 4 - DNA, 90 bp, Unknown
  artificial oligonucleotide
atgttcagcttgaaagcattattgccattggccttgttgttggtcagcgccaaccaagttgctgcaaaagtcgatgataagctgtcaaac

SEQ ID NO: 5 - DNA, 25 bp, Unknown
  artificial oligonucleotide
tgatttcaaatgtttctagagcgca

SEQ ID NO: 6 - DNA, 20 bp, Unknown
  artificial oligonucleotide
aatgctgaaattggggccaa

SEQ ID NO: 7 - DNA, 25 bp, Unknown
  artificial oligonucleotide
gcgttcaagtaatttgtcaatggaa

SEQ ID NO: 8 - DNA, 23 bp, Unknown
  artificial oligonucleotide
tttgagaagcctaccacgtaagg

SEQ ID NO: 9 - DNA, 22 bp, Unknown
  artificial oligonucleotide
tacaatcgatagattgtcgcac

SEQ ID NO: 10 - DNA, 20 bp, Unknown
  artificial oligonucleotide
agtcgtcactcatggtgatt

SEQ ID NO: 11 - DNA, 25 bp, Unknown
  artificial oligonucleotide
tgatttcaaatgtttctagagcgca

SEQ ID NO: 12 - DNA, 23 bp, Unknown
  artificial oligonucleotide
tttgagaagcctaccacgtaagg